(19) United States
(12) Patent Application Publication
(10) Pub. No.: US 2009/0312387 A1
(43) Pub. Date: Dec. 17, 2009

(54) COMPOSITION FOR CONTROLLING PARASITES ON ANIMALS

(75) Inventors: Kirkor Sirinyan, Bergisch Gladbach (DE); Andreas Turberg, Haan (DE)

Correspondence Address: BAYER HEALTHCARE LLC, P.O.BOX 390, SHAWNEE MISSION, KS, 66201, US

(73) Assignee: BAYER ANIMAL HEALTH GMBH, LEVERKUSEN (DE)

(21) Appl. No.: 12/520,169

(22) PCT Filed: Dec. 14, 2007
(86) PCT No.: PCT/EP2007/010980

§ 371 Date: Jun. 19, 2009

(30) Foreign Application Priority Data
Dec. 27, 2006 (DE) 10 2006 061 537.9

(51) Int. Cl. A61K 31/415; A61P 33/00

(52) U.S. Cl. 514/407

(57) ABSTRACT

The invention relates to novel compositions for controlling parasites on animals, comprising an N-phenylpyrazole in a formulation comprising aliphatic cyclic carbonates.

The invention relates to novel compositions for controlling parasites on animals, comprising an N-phenylpyrazole in a formulation comprising aliphatic cyclic carbonates.

N-Phenylpyrazoles and their good insecticidal and acaricidal activity are known from US 2006014802 A1, WO2005090313 A1, FR2834288A1, WO09828277, US06069157, WO200031043, DE19824487, WO09804530, WO09962903, EP00933363, EP00911329, WO09856767, US05814652, WO09845274, WO9840359, WO09828279, WO09828278, DE19650197, WO09824767, EP00846686, EP00839809, WO09728126, EP00780378, GB02308365, US05629335, WO09639389, US05556873, EP00659745, US05321040, EP00511845, EP-A-234119, EP-A-295117 and WO 98/24769. In spite of this abundance of applications with numerous N-phenylpyrazole structures, there is a superior structure type which, for most indications, shows, by comparison, the best activity. 1-[2,6-Dichloro-4-(trifluoromethyl)phenyl]-3-cyano-4-[(trifluoromethyl)-sulphinyl]-5-aminopyrazole (INN: fipronil) is generally acknowledged to be the most effective compound of this class for controlling most parasites.

N-Phenylpyrazoles have been marketed as ectoparasiticides for more than 10 years (Hunter, J. S., III, D. M. Keister and P. Jeannin. 1994. Fipronil: A new compound for animal health. Proc. Amer. Assoc. Vet. Parasitol. 39th Ann. Mtg. San Francisco, Calif. Pg. 48.). They are distinguished by good and broad activity and acceptable compatibility. It is known that the existing formulations having a high content of DEE (Transcutol) contain a strong transdermal (FR 1996-11446 A; Sicherheitsdatenblatt [Safety data sheet]: ISO/DIS 11014/29 CFR 1910.1200/ANSI Z400.1 Printing date Oct. 23, 2001: FRONTLINE® TOP SPOT™: fipronil 9.7% w/w) component. This facilitates, via the formulation, penetration into the sebaceous glands and the epithelium (Skin distribution of fipronil by microautoradiography following topical administration to the beagle dog. Cochet, Pascal; Birckel, P.; Bromet-Petit, M.: Bromet, N.: Weil, A.; European Journal of Drug Metabolism and Pharmacokinetics (1997), 22(3), 211-216.). Via sebum excretion from the sebaceous glands, a high concentration in the sebaceous glands may contribute to a long-lasting availability of the active compound If the active compound is carried along. However, in the case of the customary formulations, penetration of N-phenylpyrazoles into the circulation is also likely, since each hair follicle is supplied by a blood vessel and the follicles are thus separated from the circulation only by a very thin area (Transfollicular drug delivery—Is it a reality? Meidan, Victor M.; Bonner, Michael C.; Michniak, Bozena B.; International Journal of Pharmaceutics (2005), 306(1-2), 1-14). Thus, the availability of the active compound on the animal is limited, to, both with respect to duration and concentration, since the active compound passes into the circulation and its available concentration in the sebum is lowered accordingly.

This disadvantage of the formulation of the prior art was to be reduced by modifying the basic properties of the formulation without losing the positive efficacy properties. To this end, by intensive analyses and test series, we have now surprisingly identified, from a large number of additives, solvents and spreading agents, an additive which can improve the good arthropodicidal efficacy properties of the N-phenylpyrazoles.

The invention relates to novel compositions for controlling parasites on animals, comprising an N-phenylpyrazole in a formulation comprising:
an aliphatic cyclic carbonate
an aliphatic cyclic or acyclic polyether.

The arthropodicidal compositions according to the invention are novel and, compared to the formulations hitherto described, have considerably better and longer-lasting efficacy, with simultaneously improved user and target animal safety profile.

To the person skilled in the art, N-phenylpyrazoles are known per se as arthropodicidally active compounds, for example from the documents mentioned above, which are incorporated herein by way of reference.

Preferred phenylpyrazoles are those of the formula (I):

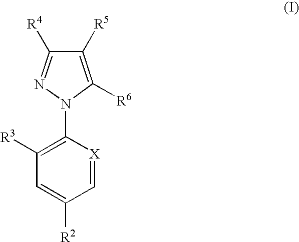

in which
X represents ═N— or C—R¹,
R¹ and R³ independently of one another represent halogen,
R² represents halogen, C₁₋₃-haloalkyl, S(O)ₙCF₃ or SF₅,
n represents 0, 1 or 2,
R⁴ represents hydrogen, cyano or a radical of the formula

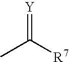

or one of the cyclic substituents below:

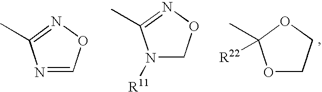

R⁵ represents hydrogen, C₂₋₄-alkynyl, C₂₋₄-alkenyl which may optionally be mono- or polysubstituted by halogen or C₁₋₃-alkyl, or R⁵ represents C₁₋₄-alkyl-(C═O)—, C₁₋₄-alkyl-S—, C₁₋₄-haloalkyl-S—, —S(═O)—C₁₋₄-alkyl or —S(═NH)—C₁₋₄-alkyl, optionally halogen-substituted phenyl, optionally halogen-substituted furyl, the radical —NR¹⁴R¹⁵, an oxiranyl radical which is optionally mono- or polysubstituted by C₁₋₄-alkyl or C₁₋₄-haloalkyl, or a cyclopropyl radical which is optionally mono- or polysubstituted by halogen, C₁₋₄-alkyl or C₁₋₄-haloalkyl,
R⁶ represents hydrogen, C₁₋₄-alkylcarbonyl or a radical —NR¹⁶R¹⁷,
R⁷ represents hydrogen, C₁₋₄-alkyl, C₁₋₄-alkyl-S— or —NR⁹R¹⁰,
Y represents ═S, ═O, ═NH, ═N—C₁₋₄-alkyl, ═N—OH or

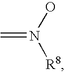

R⁸ represents C₁₋₄-alkyl,
R⁹ and R¹⁰ independently of one another represent hydrogen, hydroxyl or C₁₋₄-alkyl,
R¹¹ represents hydrogen, C₁₋₄-alkyl, —COO—C₁₋₄-alkyl or —CONR¹²R¹³,
R¹² and R¹³ independently of one another represent hydrogen or C₁₋₄-alkyl,
R¹⁴ and R¹⁵ independently of one another represent hydrogen, C₁₋₄-alkyl, C₁₋₄-haloalkyl or C₁₋₄-alkyl-SO₂—,
R¹⁶ and R¹⁷ independently of one another represent hydrogen, C₁₋₄-alkoxy or C₁₋₄-alkyl, where the C₁₋₄-alkyl may optionally be substituted by phenyl, pyranzinyl or pyridyl, where phenyl, pyranzinyl or pyridyl may be mono- or polysubstituted by hydroxyl, C₁₋₄-alkyl, C₁₋₄-haloalkyl and/or C₁₋₄-alkoxy, or
R¹⁶ and R¹⁷ represent C₁₋₄-alkylcarbonyl, C₁₋₄-alkoxycarbonyl, C₁₋₄-alkoxy-C₁₋₄-alkylcarbonyl or the radical —(C═O)NR²⁰R²¹ or
R¹⁶ and R¹⁷ together represent the group ═CR¹⁸R¹⁹ which is attached by a double bond to the nitrogen,
R¹⁸ and R¹⁹ independently of one another represent phenyl which is optionally mono- or polysubstituted by hydroxyl, C₁₋₄-alkyl, C₁₋₄-haloalkyl and/or C₁₋₄-alkoxy, and/or R¹⁸ and R¹⁹ represent hydrogen, C₁₋₄-alkyl, C₁₋₄-alkenyl or C₁₋₄-alkoxy, where C₁₋₄-alkyl, C₁₋₄-alkenyl or C₁₋₄-alkoxy may optionally be substituted by phenyl which is optionally mono- or polysubstituted by hydroxyl, C₁₋₄-alkyl, C₁₋₄-haloalkyl and/or C₁₋₄-alkoxy,
R²⁰ and R²¹ independently of one another represent hydrogen, C₁₋₄-alkyl or phenyl which is optionally mono- or polysubstituted by hydroxyl, C₁₋₄-alkyl, C₁₋₄-haloalkyl and/or C₁₋₄-alkoxy,
R²² represents C₁₋₄-alkyl.

Halogen preferably represents fluorine, chlorine, bromine or iodine, in particular fluorine, chlorine or bromine.

C₁₋₄-Alkyl represents straight-chain or branched alkyl having 1 to 4 carbon atoms, such as, for example, methyl, ethyl, n-propyl, isopropyl, n-butyl, sec-butyl, tert-butyl.

C₁₋₄-Haloalkyl represents straight-chain or branched alkyl having 1 to 4 carbon atoms which is substituted by one or more identical or different halogen atoms; this also includes perhaloalkyl compounds. Preference is given to fluoroalkyls. Examples are —CF₂H, —CF₃, —CH₂CF₃, —CF₂CF₃.

Preferably, the substituents have the following meanings:
X preferably represents C—R¹.
R¹ and R³ independently of one another preferably represent chlorine or bromine.
R² preferably represents C₁₋₃-haloalkyl or SF₅.
R⁴ preferably represents hydrogen, cyano or a radical of the formula

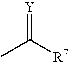

or one of the cyclic substituents below:

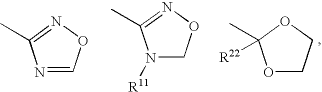

R⁵ preferably represents hydrogen, C₂₋₃-alkynyl, C₂₋₃-alkenyl which may optionally be monosubstituted by halogen or C₁₋₃-alkyl, or R⁵ preferably represents C₁₋₃-alkyl-(C═O)—, C₁₋₃-alkyl-S—, C₁₋₃-haloalkyl-S—, —S(═O)—C₁₋₃-alkyl or —S(═NH)—C₁₋₃-alkyl, optionally halogen-substituted phenyl, optionally halogen-substituted furyl, the radical —NR¹⁴R¹⁵, an optionally C₁₋₃-haloalkyl-substituted oxiranyl radical or a cyclopropyl radical which is optionally mono- or polysubstituted by halogen, C₁₋₄-alkyl or C₁₋₄-haloalkyl.
R⁶ preferably represents hydrogen, C₁₋₃-alkylcarbonyl or a radical —NR¹⁶R¹⁷.
R⁷ preferably represents hydrogen, C₁₋₄-alkyl, C₁₋₄-alkyl-S— or —NR⁹R¹⁰.
Y preferably represents ═S, ═O, ═NH, ═N—OH or

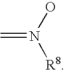

R⁸ preferably represents C₁₋₃-alkyl.
R⁹ and R¹⁰ independently of one another preferably represent hydrogen, hydroxyl or C₁₋₃-alkyl.
R¹¹ preferably represents hydrogen, C₁₋₄-alkyl or —CONR¹²R¹³.
R¹² and R¹³ independently of one another preferably represent hydrogen or C₁₋₃-alkyl.
R¹⁴ and R¹⁵ independently of one another preferably represent hydrogen, C₁₋₃-alkyl, C₁₋₃-haloalkyl or C₁₋₃-alkyl-SO₂—.
R¹⁶ and R¹⁷ independently of one another preferably represent hydrogen, C₁₋₃-alkoxy or C₁₋₃-alkyl, where the C₁₋₃-alkyl may optionally be substituted by phenyl, pyrazinyl or pyridyl, where phenyl, pyrazinyl or pyridyl may be mono- or disubstituted by hydroxyl, C₁₋₃-alkyl, C₁₋₃-haloalkyl and/or C₁₋₃-alkoxy, or
R¹⁶ and R¹⁷ represent C₁₋₄-alkylcarbonyl, C₁₋₄-alkoxycarbonyl, C₁₋₄-alkoxy-C₁₋₄-alkylcarbonyl or the radical —(C═O)NR²⁰R²¹ or
R¹⁶ and R¹⁷ together represent the group ═CR¹⁸R¹⁹ which is attached by a double bond to the nitrogen.
R¹⁸ and R¹⁹ independently of one another preferably represent phenyl which is optionally mono- or disubstituted by hydroxyl, C₁₋₃-alkyl, C₁₋₃-haloalkyl and/or C₁₋₃-alkoxy, and/or R¹⁸ and R¹⁹ represent hydrogen, C₁₋₃-alkyl, C₁₋₃-alkenyl or C₁₋₃-alkoxy, where C₁₋₃-alkyl, C₁₋₃-alkenyl or C₁₋₃-alkoxy may optionally be substituted by phenyl which is optionally mono- or disubstituted by hydroxyl, C₁₋₄-alkyl, C₁₋₄-haloalkyl and/or C₁₋₄-alkoxy.
R²⁰ and R²¹ independently of one another preferably represent C₁₋₃-alkyl or phenyl which is optionally mono- or disubstituted by hydroxyl, C₁₋₃-alkyl, C₁₋₃-haloalkyl and/or C₁₋₃-alkoxy.
R²² preferably represents C₁₋₃-alkyl.

Particularly preferably, the substituents in formula (I) have the meaning below:
X represents C—R¹.
R¹ and R³ each represent Cl.
R² represents CF₃.
R⁴ represents CN, —C(═S)NH₂ or —C(═O)CH₃.
R⁵ represents —SCHF₂, —S(═O)CF₃, —S(═O)CH₃, —S(═O)CH₂CH₃ or represents the 1-trifluoromethyloxiranyl radical.
R⁶ represents an amino group or one of the radicals below

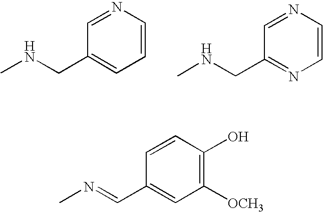

Preferred examples of compounds which can be used according to the invention are listed below:

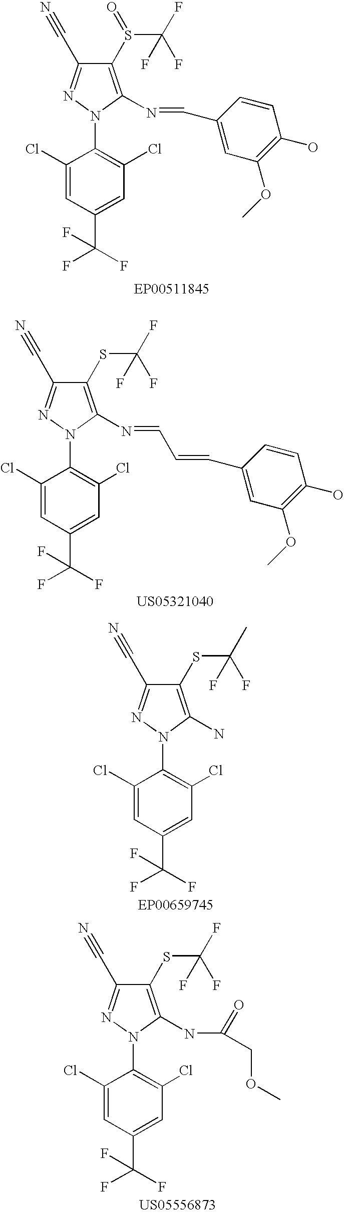

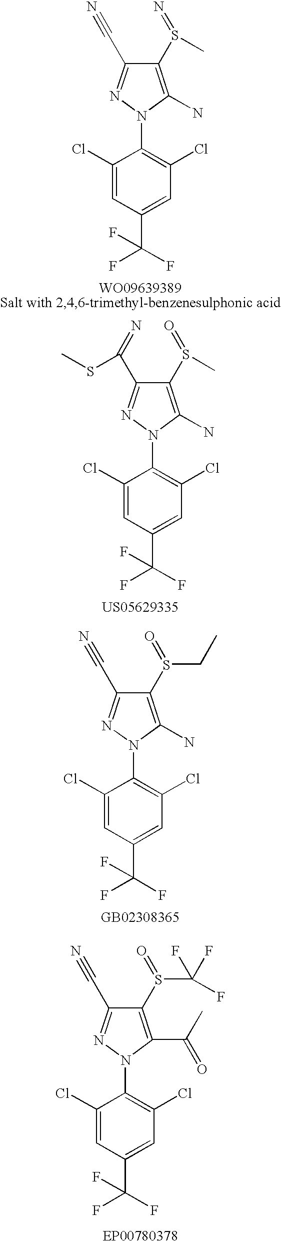

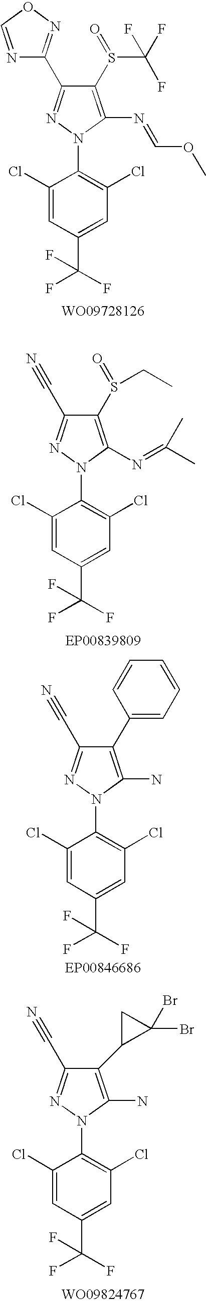

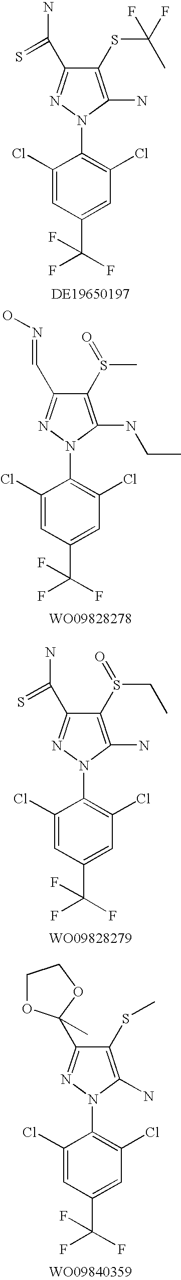

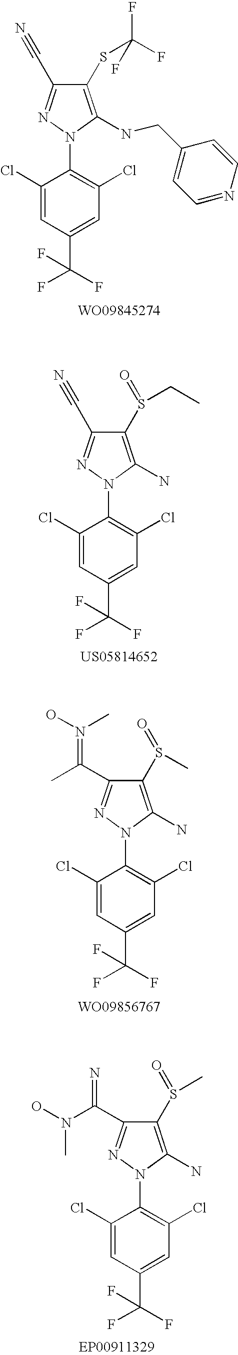

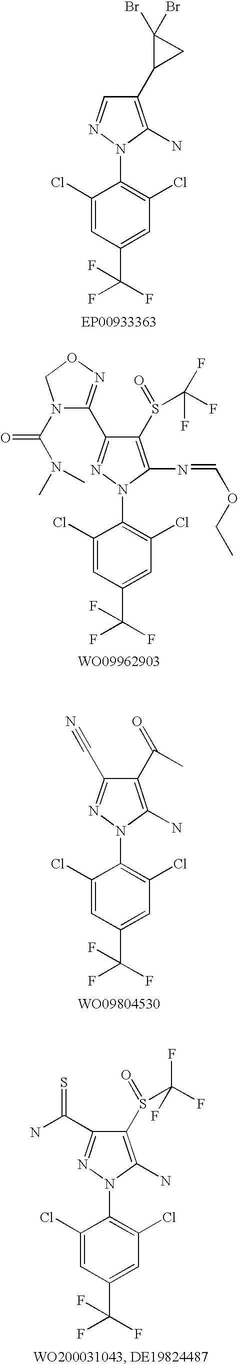

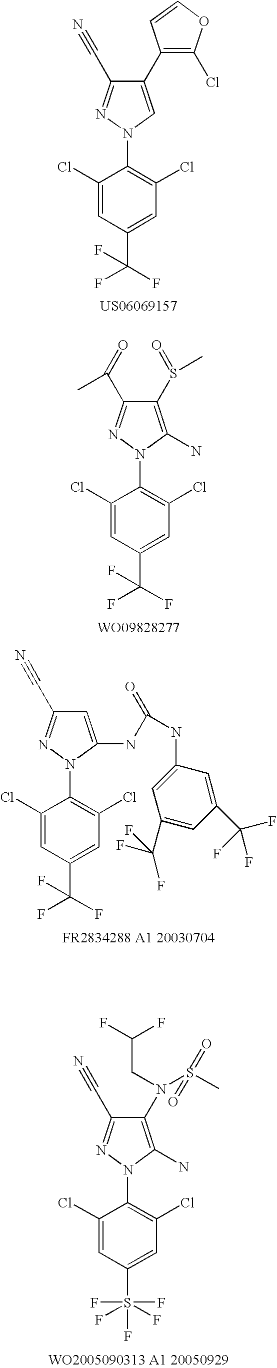

Particularly preferred examples of compounds which can be used according to the invention are:

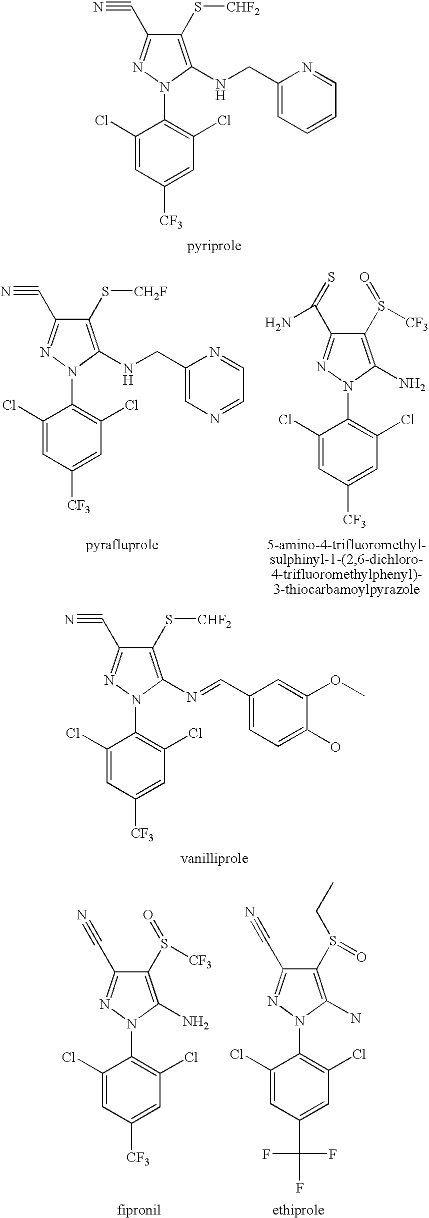

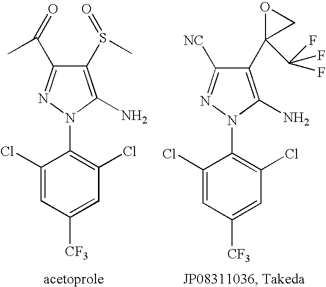

An example of a very particularly preferred N-arylpyrazole is fipronil.

A further example of a very particularly preferred N-arylpyrazole is 5-amino-4-trifluoromethylsulphinyl-1-(2,6-dichloro-4-trifluoromethylphenyl)-3-thio-carbamoylpyrazole.

Depending on the nature and arrangement of the substituents, the active compounds may, if appropriate, be present in various stereoisomeric forms, in particular as enantiomers and racemates. According to the invention, it is possible to use both the pure stereoisomers and mixtures thereof.

If appropriate, the active compounds can also be employed in the form of their salts, pharmaceutically acceptable acid addition salts and basic salts being suitable.

Suitable pharmaceutically acceptable salts are salts of mineral acids or organic acids (for example carboxylic acids or sulphonic acids). Examples which may be mentioned are salts of hydrochloric acid, sulphuric acid, acetic acid, glycolic acid, lactic acid, succinic acid, citric acid, tartaric acid, methanesulphonic acid, 4-toluenesulphonic acid, galacturonic acid, gluconic acid, embonic acid, glutamic acid or aspartic acid. Suitable pharmaceutically acceptable basic salts are, for example, the alkali metal salts, for example the sodium or potassium salts, and the alkaline earth metal salts, for example the magnesium or calcium salts.

It is furthermore also possible to use the active compounds in the form of their solvates, in particular hydrates. Solvates are to be understood as meaning both the solvates, in particular hydrates, of the active compounds themselves and the solvates, in particular hydrates, of their salts.

As solids, the active compounds may, in certain cases, form various crystal modifications. Advantageous for the use in medicaments are stable modifications having suitable solubility properties.

Unless indicated otherwise, percentages are to be understood as percent by weight based on the weight of the finished preparation.

Usually, the compositions comprise the arylpyrazole in amounts of from 1 to 27.5% by weight, preferably from 5 to 20% by weight, particularly preferably from 7.5 to 15% by weight.

The aliphatic cyclic carbonate is preferably ethylene carbonate or propylene carbonate, it also being possible to use mixtures.

The amount of aliphatic cyclic carbonate in the formulation can be varied widely in the range of from 10% by weight to 70% by weight, preferably from 12.5 to 50% by weight, particularly preferably from 15 to 40% by weight.

Aliphatic cyclic and/or acyclic ethers are compounds known per se. Preferably, they are ethers derived from diols having up to 8 carbon atoms, such as, for example, ethylene glycol, diethylene glycol, propylene glycol, dipropylene glycol. In the acyclic ethers, one or both OH groups carry a C₁₋₄-alkyl group, preferably, only one OH group is etherified; particularly preferred examples are: diethylene glycol monoethyl ether, diethylene glycol monopropyl ether, dipropylene glycol monopropyl ether. Preferred 5- or 6-membered cyclic ethers have a ring oxygen and 4 or 5 ring carbon atoms and optionally carry a C₁₋₄-alkyl substituent; preferably, they carry a free OH group either directly on the ring or on the C₁₋₄-alkyl substituent. A particularly preferred example is tetrahydrofurfuryl alcohol. The amount of aliphatic, cyclic and/or acyclic ether in the compositions according to the invention can be varied within wide limits of from 20 to 77.5% by weight, with amounts in the range of from 25 to 65% by weight and amounts in the range of from 25 to 50% by weight being particularly preferred and very particularly preferred, respectively.

According to a preferred embodiment, the compositions according to the invention may additionally comprise one or more esters of a dihydric or trihydric alcohol having up to three carbon atoms with organic fatty acids having 6 to 18 carbon atoms. As alcohol component, the esters used according to the invention contain a di- or trihydric alcohol having up to three carbon atoms, such as, for example, ethylene glycol, propylene glycol or glycerol. In general, at least two, preferably all, hydroxyl groups of the alcohol are esterified. The acid components of the esters are fatty acids having 6 to 18 carbon atoms, which may be straight-chain, branched and also mono- or polyunsaturated. It is possible to use mixed esters or else mixtures of various types of esters. Preferred triglycerides are caprylic/caprinic acid triglycerides and also carprylic/caprinic/linoleic acid triglycerides. Preference is likewise given to esters of propylene glycol with caprylic and/or caprinic acid (propylene glycol octanoate decanoate). Particularly preferably, these glycerol or propylene glycol esters of caprylic/caprinic acid have a viscosity range (20° C.) of 0.08-1.3 Pa·s, and preferably 0.08-0.40 Pa·s. It is also possible to use their polyethylene oxide-, polypropylene oxide- and/or propylene carbonate-modified derivatives having the viscosity range mentioned. Examples which may be mentioned are propylene glycol dicaprylate, propylene glycol octanoate decanoate having a viscosity range of 0.09-0.12 Pa·s, caprylic/caprinic diglyceryl succinate having a mean viscosity of 0.23 Pa·s, medium-chain caprylic/caprinic triglycerides having a viscosity of 0.27-0.30 Pa·s.

The liquid formulations according to the invention may comprise one or more of the esters mentioned above. Usually, the compositions according to the invention comprise the ester or the ester mixture in proportions of from 0 to 40% by weight, preferably from 1 to 35% by weight, particularly preferably from 1 to 12.5% by weight and very particularly preferably from 2.5 to 7.5% by weight.

If appropriate, customary organic or inorganic antioxidants may be used for stabilizing the formulations mentioned. Suitable inorganic antioxidants are, for example, the sulphites and bisulphites, in particular sodium bisulphite. Preference is given to phenolic antioxidants, such as anisole, butylated hydroxytoluene and hydroxyanisole, and their mixtures with one another. Usually, from 0.01 to 1% by weight, preferably from 0.05% to 0.5%, particularly preferably from 0.075 to 0.2% by weight are used.

The formulation ingredients mentioned, in particular the organic esters, may be stabilized against possible hydrolytic degradation using acidifying agents. Suitable acidifying agents are pharmaceutically acceptable acids, in particular carboxylic acids, such as, for example, succinic acid, tartaric acid, lactic acid or citric acid. Their preferred amount is in the range of from 0 to 0.5% by weight, but preferably from 0 to 0.2% by weight.

Polymeric surfactants based on polymethoxysiloxanes having a low surface tension of <30 mN/m, preferably <22 mN/m, can be used as further formulation auxiliaries for improving the spreadability. Such surfactants are known ethoxylated and/or propoxylated, preferably neutral or particularly preferably cationic formulation auxiliaries. An example of a preferred polymeric auxiliary which may be mentioned is the methoxysilane/ethylene oxide copolymer Belisil Silvet L 77 from Bayer GE Siliconics GmbH. The amount of these formulation auxiliaries may be varied within wide limits in the range of from 0.01 to 1.0% by weight. The preferred range is from 0.2 to 0.4% by weight.

If appropriate, the formulations may comprise further pharmaceutically acceptable auxiliaries and additives.

The compositions according to the invention may also comprise one or more further active compounds as combination partners for the arylpyrazoles. Preferred examples of such active compounds for combinations which may be mentioned are: growth inhibitors, such as, for example, chitin biosynthesis inhibitors, such as, for example, benzoylphenylureas (for example triflumuron, lufenuron); phenyloxazolines (for example etoxazole); juvenile hormone analogues (for example methoprene, hydroprene, pyriproxifen) and also mixtures of these active compounds with one another. Their amount may be varied within wide limits in the range of from 0.1 to 7.5% by weight, but preferably from 0.25 to 5.0% by weight, particularly preferably from 0.25 to 2.5% by weight.

The formulations according to the invention may also comprise synergists. Synergists in the sense of this application are to be understood as meaning compounds which for their part do not have the desired activity, but which, as mixing partners, increase the activity of the active compounds. Piperonyl butoxide, MG 264, verbutin, S,S,S-tributyl phosphorotrithioate may be mentioned here in an exemplary manner.

The compositions according to the invention are environmentally compatible and have a low toxicity which is reduced compared to that of known compositions. Accordingly, they are user-friendly and furthermore distinguished by their easy handling. The compositions have a favourable flashpoint of >70° C. and can therefore be manufactured in simple plants which do not require additional measures to protect against explosions.

Having favourable homeotherm toxicity, the compositions of the invention are suitable for controlling parasitic arthropods, in particular insects and arachids, very particularly fleas and ticks, encountered on animals, in particular homeotherms, particularly preferably mammals. These animals may be domestic animals and useful animals and also zoo animals, laboratory animals, test animals and pets.

The compositions described herein are used in particular against ectoparasites on pets and useful animals.

The compositions of the invention are active against all or individual stages of development of the pests and against resistant and normally sensitive pest species.

The pests include:

from the order of the Anoplura, for example, Haematopinus spp., Linognathus spp., Solenopotes spp., Pediculus spp., Pthirus spp.;

from the order of the Mallophaga, for example, Trimenopon spp., Menopon spp., Eomenacanthus spp., Menacanthus spp., Trichodectes spp., Felicola spp., Damalinea spp., Bovicola spp.;
from the order of the Diptera, suborder Brachycera, for example, Chrysops spp., Tabanus spp., Musca spp., Hydrotaea spp., Muscina spp., Haematobosca spp., Haematobia spp., Stomoxys spp., Fannia spp., Glossina spp., Lucilia spp., Calliphora spp., Auchmeromyia spp., Cordylobia spp., Cochliomyia spp., Chrysomyia spp., Sarcophaga spp., Wohlfartia spp., Gasterophilus spp., Oesteromyia spp., Oedemagena spp., Hypoderma spp., Oestrus spp., Rhinoestrus spp., Melophagus spp., Hippobosca spp.;
from the order of the Diptera, suborder Nematocera, for example, Culex spp., Aedes spp., Anopheles spp., Culicoides spp., Phlebotomus spp., Simulium spp.;
from the order of the Siphonaptera, for example, Ctenocephalides spp., Echidnophaga spp., Ceratophyllus spp., Pulex spp.;
from the order of the Metastigmata, for example, Hyalomma spp., Rhipicephalus spp., Boophilus spp., Amblyomma spp., Haemaphysalis spp., Dermacentor spp., Ixodes spp., Argas spp., Ornithodorus spp., Otobius spp.;
from the order of the Mesostigmata, for example, Dermanyssus spp., Ornithonyssus spp., Pneumonyssus spp.;
from the order of the Prostigmata, for example, Cheyletiella spp., Psorergates spp., Myobia spp., Demodex spp., Neotrombicula spp.;
from the order of the Astigmata, for example, Acarus spp., Myocoptes spp., Psoroptes spp., Chorioptes spp., Otodectes spp., Sarcoptes spp., Notoedres spp., Knemidocoptes spp., Neoknemidocoptes spp., Cytodites spp., Laminosioptes spp.;

Particular emphasis may be given to the action against fleas (Siphonaptera for example, Ctenocephalides spp, Echidnophaga spp., Ceratophyllus spp., Pulex spp.), ticks (Hyalomma spp., Rhipicephalus spp., Boophilus spp., Amblyomma spp., Haemaphysalis spp., Dermacentor spp., Ixodes spp., Argas spp., Ornithodorus spp., Otobius spp.) and the Diptera mentioned above (Chrysops spp., Tabanus spp., Musca spp., Hydrotaea spp., Muscina spp., Haematobosca spp., Haematobia spp., Stomoxys spp., Fannia spp., Glossina spp., Lucilia spp., Calliphora spp., Auchmeromyia spp., Cordylobia spp., Cochliomyia spp., Chrysomyia spp., Sarcophaga spp., Wohlfartia spp., Gasterophilus spp., Oesteromyia spp., Oedemagena spp., Hypoderma spp., Oestrus spp., Rhinoestrus spp., Melophagus spp., Hippobosca spp.).

The useful and breeding animals include mammals, such as, for example, cattle, horses, sheep, pigs, goats, camels, water buffalo, donkeys, rabbits, fallow deer, reindeer, fur-bearing animals, such as, for example, mink, chinchilla, raccoon, birds, such as, for example, hens, geese, turkeys, ducks.

The laboratory animals and test animals include mice, rats, guinea pigs, rabbits, golden hamsters, dogs and cats.

The pets include dogs and cats.

Particular emphasis is given to application on cat and dog.

Application can take place both prophylactically and therapeutically.

Preferably, the liquid formulations according to the invention are suitable for spot-on, pour-on or spray application, where the spray application may be carried out, for example, using a pump spray or an aerosol spray (pressurized spray). For specific indications, the formulations may also be used after dilution with water as a dip; in this case, the formulation should contain emulsifying additives.

The preferred application forms are pump spray, pour-on and spot-on. The spot-on application is very particularly preferred.

The formulations according to the invention are distinguished by excellent compatibility with customary “single-dose” plastic tubes and by their storage stability in various climate zones. They have low viscosity and can be applied without any problems.

The liquid formulations according to the invention can be prepared by mixing the appropriate amounts of the components with one another, using, for example, conventional stirring tanks or other suitable instruments. If required by the ingredients, it is also possible to operate under a protective atmosphere or with other methods of excluding oxygen.

### EXAMPLES

### Example 1

100 ml of liquid formulation consisting of
10.0 g of 5-amino-4-trifluoromethylsulphinyl-1-(2,6-dichloro-4-trifluoromethyl-phenyl)-3-thiocarbamoylpyrazole
72.7 g of diethylene glycol monoethyl ether
25.0 g of propylene carbonate
5.0 g of propylene glycol octanoate decanoate
0.1 g of butylated hydroxytoluene
0.2 g of butylated hydroxyanisole

### Example 2

100 ml of liquid formulation consisting of
10.5 g of fipronil
57.75 g of dipropylene glycol monomethyl ether
40.0 g of propylene carbonate
5.0 g of propylene glycol octanoate decanoate
0.1 g of butylated hydroxytoluene
0.2 g of butylated hydroxyanisole

### Example 3

100 ml of liquid formulation consisting of
10.5 g of fipronil
72.75 g of dipropylene glycol monomethyl ether
25.0 g of propylene carbonate
5.0 g of propylene glycol octanoate decanoate
0.1 g of butylated hydroxytoluene
0.2 g of butylated hydroxyanisole

### Example 4

100 ml of liquid formulation consisting of
10.0 g of 5-amino-4-trifluoromethylsulphinyl-1-(2,6-dichloro-4-trifluoromethyl-phenyl)-3-thiocarbamoylpyrazole
57.7 g of diethylene glycol monoethyl ether
40.0 g of propylene carbonate
5.0 g of propylene glycol octanoate decanoate
0.1 g of butylated hydroxytoluene
0.2 g of butylated hydroxyanisole

### Example 5

100 ml of liquid formulation consisting of
11.4 g of fipronil
60.0 g of diethylene glycol monoethyl ether
25.0 g of propylene carbonate
5.0 g of propylene glycol octanoate decanoate
0.12 g of butylated hydroxytoluene
0.2 g of butylated hydroxyanisole
0.25 g of Silvet L 77 from Bayer GE Siliconics GmbH

### Comparative Example

A commercially available 10% fipronil spot-on formulation from Merial Ltd., 3239 Satellite Blvd., Duluth, Ga. 30096-4640, USA.

### Biological Examples

The tested formulations were metered out exactly by weight to ensure better comparability. To this end, 20 pipettes of the fipronil-containing commercial preparation (comparative example) were emptied into a glass bottle and likewise blinded using a code.

All samples were applied as a single spot to the neck (cats and smaller dogs) using Eppendorf pipettes (volume up to 0.95 ml). For application volumes of more than 1 ml, the volume was halved and applied to the neck as two spots at a distance of about 10 cm.

Further laboratory tests for the activity against fleas and ticks according to Examples 2 and 4 show that the preparations in the abovementioned formulations according to the invention have very good and long-lasting action against ticks and fleas which, in the tests, is consistently superior to the prior art. Furthermore, the preparations in the abovementioned formulations according to the invention are distinguished in that they are tolerated by target animal and user, and they are thus highly suitable for controlling fleas and ticks on small animals. Thus, for example, a formulation according to Example 2 and a formulation according to Example 4 are, after oral ingestion, 2× and 3× better tolerated, respectively, than formulations of the prior art.

A. Activity Against Fleas (Ctenocephalides felis) on Dogs

Between days-4 and -1, dogs are infested 1-2 times with about 100 adult unfed Ctenocephalides felis per dog. The fleas are placed on the neck of the animal.

On day 0, the success of the infestation on the dog is examined by checking the awake animals for fleas. The number of live fleas is noted.

After the fleas have been counted, the animals are treated. The dogs of the control group are not treated. The medicaments to be examined are administered to the animals dermally as a spot-on in an application rate of 0.1-0.15 ml/kg of bodyweight or as a spray in an application rate of 1-1.5 ml/kg of bodyweight. The application is carried out once on day 0. Only animals that are clinically healthy are used.

On days 1 and 2, all dogs are examined for live fleas. The results are noted with the crude data.

On days 7, 14, 21, 28 and 35 and, if appropriate, also on days 42 and 49, all dogs are reinfested with about 100 adult unfed Ctenocephalides felis per dog. In each case one day after the reinfestation, all dogs are checked for live fleas. The results are noted with the crude data.

A formulation is considered to be highly effective if, between 24 and 48 hours after reinfestation, an efficacy of >95% is found, and this action persists for at least 3-4 weeks.

The efficacy is calculated using a modified formula according to Abbott:

$$Efficacy \% = \frac{number of fleas CG - number of fleas TG}{number of fleas CG} \times 100$$

$$CG \text{:} control group ;$$

$$TG \text{:} treatment group$$

The medicaments of Formulation Examples 2 and 4, applied as a spot-on at a dosage of 0.1 and 0.15 ml/kg, respectively, were found to be highly effective against Ctenocephalides felis.

B. Activity Against Ticks (Rhipicephalus sanguineus, Dermacentor variabilis) on Dogs

Between days-4 and -1, dogs are sedated using 2% Rompun® (Bayer AG, active compound: xylazine hydrochloride) (0.1 ml/kg of bodyweight). Once all dogs have been sedated (after about 10-15 minutes), they are transferred to transport boxes, and 50 Rhipicephalus sanguineus or Dermacentor variabilis (25 ♀, 25♂) per dog are applied to the neck of the animal. After about 1½ hours, the animals are retransferred from the transport box into the cage.

On day 0, the success of the infestation on the dog is examined by checking the awake animal for ticks. An intensive search is carried out in the region of the head and the ears, including the folds of the ears, in the region of the neck, on the lower abdomen, on the lower breast, on the flank and in between the toes and the limbs. The number of sucking live ticks is noted. Dead ticks are removed.

After the ticks have been counted, the animals are treated. The dogs of the control group are not treated. The medicaments to be examined are administered to the animals dermally as a spot-on at 0.1-0.15 ml/kg of bodyweight or as a spray at 1-1.5 ml/kg of bodyweight. The application is carried out once on day 0. Only animals which are clinically healthy are used.

On day 1 and day 2, all dogs are checked for living and dead sucking ticks. The results are noted with the crude data. On day 2, all living and dead ticks are removed from the dog.

On days 7, 14, 21, 28, 35 and, if appropriate, also on days 42 and 49, all dogs are reinfested with in each case 50 Rhipicephalus sanguineus or Dermacentor variabilis (25♀), 25♂) per dog. In each case two days after the reinfestation, all dogs are checked for living and dead sucking ticks. The results are noted with the crude data. On the second day after the reinfestation, all living and dead ticks are removed from the dog.

A formulation is considered to be highly effective if, on day 2 and in each case on the second day after reinfestation, an efficacy of >90% is found, and this action persists for at least 3 weeks.

For calculating the efficacy, a modified formula according to Abbott is used:

$$Efficacy \% = \frac{number of ticks CG - number of ticks TG}{number of ticks CG} \times 100$$

$$CG \text{:} control group ;$$

$$TG \text{:} treatment group$$

The medicaments according to Formulation Examples 2 and 4, applied as a spot-on at a dosage of 0.1 and 0.15 ml/kg, respectively, were found to be highly effective against Rhipicephalus sanguineus.

C. Activity Against Fleas (Ctenocephalides felis) on Cats

On day-1, cats are infested with about 100 adult unfed Ctenocephalides felis per cat. The fleas are placed on the neck of the animal.

On day 0, the success of the infestation on the cat is examined by checking the awake animal for fleas. The number of live fleas is noted.

After the fleas have been counted, the animals are treated. The cats of the control group are not treated. The medicaments to be examined are administered to the animals dermally as a spot-on in an application rate of 0.1-0.15 ml/kg of bodyweight. The application is carried out once on day 0. Only animals that are clinically healthy are used.

On day 2, all cats are examined for live fleas. The results are noted with the crude data.

On days 7, 14, 21, 28 and 35 and, if appropriate, also on days 42 and 49, all cats are reinfested with about 100 adult unfed Ctenocephalides felis per cat. In each case two days after reinfestation, all cats are checked for live fleas. The results are noted with the crude data.

A formulation is considered to be highly effective if, on day 2 and in each case on the second day after reinfestation, an efficacy of >95% is found, and this action persists for at least 3-4 weeks.

The efficacy is calculated using a modified formula according to Abbott:

$$Efficacy \% = \frac{number of fleas CG - number of fleas TG}{number of fleas CG} \times 100$$

$$CG \text{:} control group ;$$

$$TG \text{:} treatment group$$

The medicaments of Formulation Examples 2 and 4, applied as a spot-on at a dosage of 0.1 and 0.15 ml/kg, respectively, were found to be highly effective against Ctenocephalides felis.

D. Activity Against Ticks (Ixodes ricinus) on Cats In each case on day-2, cats are sedated using a mild sedative (acepromazine maleate). Once all cats have been sedated (after about 10-15 minutes), 30-50 Ixodes ricinus (15-25♀, 15-25♂) per cat are applied to the neck of the animal.

On day-1, the success of the infestation on the cats is examined by checking the awake animal for ticks. An intensive search is carried out in the region of the head and the ears, in the region of the neck, on the lower abdomen, on the lower breast, on the flank and on the limbs. The number of sucking live ticks is noted. Dead ticks are removed.

After the ticks have been counted, the animals are divided into groups. Treatment is carried out on day 0. The cats of the control group are not treated. The medicaments to be examined are administered to the animals dermally, as a spot-on at 0.1-0.15 ml/kg of bodyweight. Application is carried out once on day 0. Only animals which are clinically healthy are used.

On day 2, all cats are checked for living and dead sucking ticks. The results are noted with the crude data. All living and dead ticks are removed from the cat.

On days 7, 14, 21, 28 and 35 and, if appropriate, also on days 42 and 49, all cats are reinfested with in each case 30-50 Ixodes ricinus (15-25♀), 15-25♂). In each case two days after the reinfestation, all cats are checked for living and dead sucking ticks. The results are noted with the crude data. On the second day after the reinfestation, all living and dead ticks are removed from the cat.

A formulation is considered to be highly effective if, on day 2 and in each case on the second day after reinfestation, an efficacy of >90% is found, and this action persists for at least 3 weeks.

The efficacy is calculated using a modified formula according to Abbott:

$$Efficacy \% = \frac{number of ticks CG - number of ticks TG}{number of ticks CG} \times 100$$

$$CG \text{:} control group ;$$

$$TG \text{:} treatment group$$

The medicaments according to Formulation Examples 2 and 4, applied as a spot-on at a dosage of 0.1-0.15 ml/kg, were found to be highly effective against Ixodes ricinus.

### E. Efficacy Against Fleas and Ticks Over 5 to 7 Weeks

The efficacy of the compositions according to the invention against fleas and ticks was tested over a period of five to seven weeks. The test was carried out according to the description under items A to D. The results are shown in Tables 1a, 1b, 2a, 2b and 3.

TABLE 1a
Efficacy of the composition according to Examples 2 and 4 against fleas on cats

| | T0 | Appl. Vol | | W0 | | W1 | | W2 |
|---|---|---|---|---|---|---|---|---|
| | Treatment | ml/kg | Parasite | T2 | | T9 | | T16 |
| 1. infestation day - 4 | Comparative | 0.1 | Ctenocephalides felis | 97 | 2. infestation day 7 | 100 | 3. infestation day 21 | 100 |
| | Example | | | | | | | |
| | Example 2 | 0.1 | Ctenocephalides felis | 99 | | 100 | | 100 |
| | Example 4 | 0.15 | Ctenocephalides felis | 100 | | 100 | | 100 |

| | W3 | | W4 | | W5 | | W6 |
|---|---|---|---|---|---|---|---|
| | T23 | | T30 | | T37 | | T44 |
| 4. infestation day 14 | 100 | 5. infestation day 28 | 99 | 6. Infestation day 35 | 100 | 7. Infestation day 42 | 99 |
| | 100 | | 100 | | 99 | | 100 |
| | 100 | | 100 | | 99 | | 99 |

TABLE 1b
Efficacy of the composition according to Examples 2 and 4 against ticks on cats

| | T0 | Appl. Vol | | W0 | | W1 | | W2 |
|---|---|---|---|---|---|---|---|---|
| | Treatment | ml/kg | Parasite | T2 | | T9 | | T16 |
| 1. infestation day - 4 | Comparative | 0.1 | Ixodes ricinus | 74 | 2. infestation day 7 | 99 | 3. infestation day 14 | 96 |
| | Example | | | | | | | |
| | Example 2 | 0.1 | Ixodes ricinus | 78 | | 100 | | 100 |
| | Example 4 | 0.15 | Ixodes ricinus | 74 | | 100 | | 100 |

| | W3 | | W4 | | W5 | | W6 |
|---|---|---|---|---|---|---|---|
| | T23 | | T30 | | T37 | | T44 |
| 4. infestation day 21 | 72 | 5. infestation day 28 | 82 | 6. infestation day 35 | 89 | 7. infestation day 42 | 83 |
| | 97 | | 97 | | 83 | | 70 |
| | 98 | | 80 | | 70 | | 89 |

Appl. Vol = volume applied in ml/kg of bodyweight
“value” % = efficacy in %, calculated via determination of the geometrical mean compared to an untreated control group

TABLE 2a
Efficacy of the composition according to Examples 2 and 4 against fleas on dogs

| | | T0 | Appl. Vol | | W0 | | W1 |
|---|---|---|---|---|---|---|---|
| | | Treatment | ml/kg | Parasite | T2 | | T9 |
| 1. infestation day - 4 | 2. infestation day - 1 | Example 2 | 0.1 | Ctenocephalides felis | 100 | 3. infestation day 7 | 100 |
| | | Example 4 | 0.15 | Ctenocephalides felis | 100 | | 100 |
| | | Comparative | 0.1 | Ctenocephalides felis | 100 | | 100 |
| | | Example | | | | | |

| | W2 | | W3 | | W4 | | W5 | | W6 | | W7 |
|---|---|---|---|---|---|---|---|---|---|---|---|
| | T16 | | T23 | | T30 | | T37 | | T44 | | T51 |
| 4. infestation | 100 | 5. infestation | 100 | 6. infestation | 100 | 7. Infestation | 94 | 8. Infestation | 93 | 9. Infestation | 65 |
| day 14 | 100 | day 21 | 100 | day 28 | 100 | day 35 | 100 | day 42 | 99 | day 49 | 96 |
| | 100 | | 100 | | 99 | | 98 | | 84 | | 3 |

TABLE 2b
Efficacy of the composition according to Examples 2 and 4 against ticks on dogs

| | | | Appl. | | | | |
|---|---|---|---|---|---|---|---|
| | | T0 | Vol | | W0 | | W1 |
| | | Treatment | ml/kg | Parasite | T2 | | T9 |
| 1. infestation day - 4 | 2. infestation day - 1 | Example 2 | 0.1 | Rhipicephalus sanguineus | 99 | 3. infestation day 7 | 100 |
| | | Example 4 | 0.15 | Rhipicephalus sanguineus | 88 | | 100 |
| | | Comparative | 0.1 | Rhipicephalus sanguineus | 94 | | 100 |
| | | Example | | | | | |

| | W2 | | W3 | | W4 | | W5 | | W6 | | W7 |
|---|---|---|---|---|---|---|---|---|---|---|---|
| | T16 | | T23 | | T30 | | T37 | | T44 | | T51 |
| 4. infestation | 100 | 5. infestation | 100 | 6. infestation | 100 | 7. Infestation | 100 | 8. Infestation | 100 | 9. Infestation | 97 |
| day 14 | 100 | day 21 | 100 | day 28 | 100 | day 35 | 100 | day 42 | 99 | day 49 | 87 |
| | 100 | | 100 | | 100 | | 94 | | 93 | | 65 |

Appl. Vol = volume applied in ml/kg of bodyweight
“value” % = efficacy in %, calculated via determination of the geometrical mean compared to an untreated control group

TABLE 3
Efficacy of the composition according to Examples 2 and 4 against ticks on dogs

| | T0 | Appl. Vol | | W0 | | W1 | | W2 |
|---|---|---|---|---|---|---|---|---|
| | Treatment | ml/kg | Parasite | T2 | | T9 | | T16 |
| 1. infestation day - 4 | Comparative | 0.1 | Dermacentor variabilis | 25 | 2. infestation day 7 | 98 | 3. infestation day 14 | 99 |
| | Example | | | | | | | |
| | Example 2 | 0.1 | Dermacentor variabilis | 82 | | 100 | | 100 |
| | Example 4 | 0.15 | Dermacentor variabilis | 92 | | 93 | | 100 |

| | W3 | | W4 | | W5 | | W6 |
|---|---|---|---|---|---|---|---|
| | T23 | | T30 | | T37 | | T44 |
| 4. infestation day 21 | 98 | 5. infestation day 28 | 98 | 6. infestation day 35 | 92 | 7. infestation day 42 | 91 |
| | 98 | | 98 | | 100 | | 95 |
| | 99 | | 99 | | 88 | | 92 |

Appl. Vol = volume applied in ml/kg of bodyweight
“value” % = efficacy in %, calculated via determination of the geometrical mean compared to an untreated control group

1. A composition for controlling parasites on animals, comprising:
a. an N-phenylpyrazole;
b. an aliphatic cyclic carbonate; and,
c. an aliphatic cyclic or acyclic polyether.

2. The composition of claim 1, further comprising an ester of a dihydric or trihydric alcohol having up to three carbon atoms with organic fatty acids having 6 to 18 carbon atoms.

3. The composition of claim 1, wherein the N-phenylpyrazole comprises from 1 to 27.5% by weight of the composition.

4. The composition of claim 1, wherein the aliphatic cyclic carbonate comprises from 10 to 70% by weight of the composition.

5. The composition of claim 1, wherein the aliphatic cyclic or acyclic polyether comprises from 20 to 77.5% by weight of the composition.

6. (canceled)

7. The composition of claim 1, wherein the N-phenylpyrazole is selected from the group consisting of pyriprole, pyrafluprole, 5-amino-4-trifluoromethyl-sulphinyl-1-(2,6-dichloro-4-trifluoromethylphenyl)-3-thiocarbamoylpyrazole, vanilliprole, fipronil, ethiprole, and acetoprole.

8. The composition of claim 1, wherein the N-phenylpyrazole is fipronil.

9. The composition of claim 1, wherein the N-phenylpyrazole comprises from 7.5 to 15% by weight of the composition.

10. The composition of claim 1, wherein the aliphatic cyclic carbonate is selected from the group consisting of ethylene carbonate, propylene carbonate, or mixtures thereof.

11. The composition of claim 1, wherein the aliphatic cyclic carbonate comprises from 15 to 40% by weight of the composition.

12. The composition of claim 1, wherein the aliphatic cyclic or acyclic polyether is selected from the group consisting of monoethyl ether, diethylene glycol monopropyl ether, dipropylene glycol monopropyl ether, and tetrahydrofurfuryl alcohol.

13. The composition of claim 1, wherein the aliphatic cyclic or acyclic polyether comprises from 25 to 50% by weight of the composition.

14. A method of controlling parasites in parasite infested animals, which comprises applying to said animals an effective amount of the composition of claim 1.

15. The method of claim 14, wherein the animal is selected from cats and dogs.

16. The method of claim 14, wherein the parasite is selected from the group consisting of fleas and ticks.

17. A spot-on composition for controlling parasites on animals, comprising:
a. fipronil;
b. an aliphatic cyclic carbonate; and,
c. an aliphatic cyclic or acyclic polyether
wherein the fipronil comprises 7.5 to 15% by weight of the composition, the aliphatic cyclic carbonate comprises from 15 to 40% by weight of the composition, and the aliphatic cyclic or acyclic polyether comprises from 25 to 50% by weight of the composition.

18. The composition of claim 17, wherein the aliphatic cyclic carbonate is selected from the group consisting of ethylene carbonate, propylene carbonate, or mixtures thereof.

19. The composition of claim 18, wherein the aliphatic cyclic or acyclic polyether is selected from the group consisting of monoethyl ether, diethylene glycol monopropyl ether, dipropylene glycol monopropyl ether, and tetrahydrofurfuryl alcohol.
